SEQUENCE LISTING (10 sequences)

SEQ ID NO: 1 - DNA, 19 bp, Artificial Sequence
  forward primer for OCT 4
agaaggagtggtccgagtg

SEQ ID NO: 2 - DNA, 20 bp, Artificial Sequence
  reverse primer for OCT 4
agagtggtgacggagacagg

SEQ ID NO: 3 - DNA, 20 bp, Artificial Sequence
  forward primer for Nanog
atacctcagcctccagcaga

SEQ ID NO: 4 - DNA, 20 bp, Artificial Sequence
  reverse primer for Nanog
cctgattgrrccaggattgg

SEQ ID NO: 5 - DNA, 20 bp, Artificial Sequence
  forward primer for KLF4
accctgggtcttgaggaagt

SEQ ID NO: 6 - DNA, 20 bp, Artificial Sequence
  reverse primer for KLF4
tgccttgagatgggaactct

SEQ ID NO: 7 - DNA, 21 bp, Artificial Sequence
  forward primer for Sox2
gatgcacaactcggagatcag

SEQ ID NO: 8 - DNA, 21 bp, Artificial Sequence
  reverse primer for Sox2
gccgttcatgtaggtctgcga

SEQ ID NO: 9 - DNA, 20 bp, Artificial Sequence
  forward primer for GAPDH
gaaggtgaaggtcggagtca

SEQ ID NO: 10 - DNA, 20 bp, Artificial Sequence
  reverse primer for GAPDH
ggaggcattgctgatgatct

(19) United States
(12) Patent Application Publication
(10) Pub. No.: US 2014/0227235 A1
(43) Pub. Date: Aug. 14, 2014

(54) CARTILAGE CELL TREATMENT COMPRISING COLLAGEN, HYALURONIC ACID DERIVATIVE, AND STEM CELL DERIVED FROM MAMMAL UMBILICAL CORD

(71) Applicants: Sun Mi Kim, Gyeonggi-do (KR); Youngjun Lee, Seoul (KR); Yong Soo Choi, Gyeonggi-do (KR)

(72) Inventors: Sun Mi Kim, Gyeonggi-do (KR); Youngjun Lee, Seoul (KR); Yong Soo Choi, Gyeonggi-do (KR)

(73) Assignee: CHA BIO & DIOSTECH CO., LTD., Seoul (KR)

(21) Appl. No.: 14/232,569

(22) PCT Filed: Jul. 11, 2012
(86) PCT No.: PCT/KR2012/005518

§ 371 Date: Jan. 13, 2014

(30) Foreign Application Priority Data
Jul. 13, 2011 (KR) 10-2011-0069551

(51) Int. Cl. A61K 38/39; A61K 35/48; A61K 47/36

(52) U.S. Cl. CPC A61K 38/39; A61K 47/36; A61K 35/51
USPC 424/93.7; 514/17.2

(57) ABSTRACT

The present invention relates to a medical composite biomaterial. More specifically, the present invention relates to a medical composite biomaterial including collagen and a hyaluronic acid derivative. Also, the present invention relates to a cartilage cell treating agent using the biomaterial and stem cells derived from a mammal umbilical cord. The biomaterial does not cause an immune reaction, has superior durability, and the cartilage cell treating agent comprising the biomaterial and the stem cells enables arthroscopic surgery, thereby reducing pain of the patient and effectively treat of degenerative arthritis and cartilage damage.

### RELATED APPLICATION

This application claims the benefit of Korean Patent Application No. 10-2011-0069551, filed on Jul. 13, 2011, in the Korean Intellectual Property Office, the disclosure of which is incorporated herein in its entirety by reference.

### BACKGROUND

1. Field

One or more embodiments of the present invention relate to a biomaterial including collagen and a hyaluronic acid derivative, and a cartilage cell treatment including the biomaterial and umbilical cord-derived stem cells.

2. Description of the Related Art

Collagen is the most common protein found in humans and is the most numerous protein in mammals, which comprises about 25% to about 35% of all the proteins of the body. More particularly, collagen is an important component of bones, tendons, and ligaments and primarily maintains the structure of organs. Collagen may be easily extracted from the skin of cows or pigs. However, collagen is derived from non-human animals and thus, collagen has problems with regards to immune responses when collagen is applied to a human body.

On the contrary, unlike collagen, the structure of hyaluronic acid does not differ among species ranging from bacteria to mammals and thus, hyaluronic acid does not act as an antigen. Accordingly, hyaluronic acid is currently used as a substitute filler material. The structure of hyaluronic acid is the same across all species and thus, has little immune response, which solves the problem of a collagen filler.

Hyaluronic acid decomposes through two pathways in vivo: the first pathway is via a hyaluronidase and the second pathway is through the adherence of hyaluronic acid to a cell receptor to be gulfed into a cell and then decomposed by an enzyme in a lysosome. The biodegradation of hyaluronic acid occurs very fast and it is known that hyaluronic acid is completely decomposed in about 0.5 days to about a few days. Accordingly, hyaluronic acid decomposes in vivo as time passes and thus, there are limitations to maintaining the effects of hyaluronic acid.

Meanwhile, cartilage tissue, unlike other tissues, does not have nerves and blood vessels and thus, when cartilage tissue does not self-regenerate once cartilage tissue is damaged. Accordingly, a surgery such as an artificial joint surgery, microfracture, or mosaicplasty is inevitable as a conventional treatment method. However, such method does not provide a complete solution due to problems such as durability of an implanted artificial joint that only lasts for more or less 10 years and a secondary infection due to the surgery.

Due to the recent progress in tissue engineering and regenerative medicine, a cartilage cell treatment was developed to completely regenerate cartilage tissue by using human cells (KR10-2010-0084142A). A conventional cartilage cell treatment is an autologous cartilage cell treatment, wherein a portion of cartilage tissue of a patient is taken to culture the tissue in vitro in bulk for about 4 weeks. First, a diseased area is cleaned and then periosteum tissue is extracted from a tibia of the patient, then the periosteum tissue is disposed and sealed on a cartilage area, and then cells cultured in a damaged cartilage area are transplanted in a suspended state. This is an autologous cartilage cell treatment wherein fibrin glue is applied to the area sealed to prevent the leakage of cells.

However, this method has limitations because the diseased area may not be sufficiently treated only with cultured cells when the damaged area is big and may be pressed by weight such that the cells may be leaked to prevent tissue regeneration.

While overcoming such problems and developing an effective biomaterial and a cartilage treatment, a novel biomaterial having little immune reaction and capable of maintaining effectiveness for a long time was developed. Furthermore, it has been found that stem cells may be added to the novel biomaterial to be used as a cartilage treatment to complete the present invention. Furthermore, when a cross-linked hyaluronic acid derivative and a collagen are mixed and then stem cells are mixed thereto to culture the same in vitro to prepare a collagen composite, an optimal mixture ratio of the hyaluronic acid and the collagen, in which the collagen composite does not swell or contract, was found to complete the present invention.

### SUMMARY

One or more embodiments of the present invention include a composition including collagen and hyaluronic acid or a derivative thereof, and a biomaterial composition including the composition.

One or more embodiments of the present invention include a cartilage cell treatment including collagen, hyaluronic acid or a derivative thereof, and stem cells.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 shows proliferation potency of the umbilical cord-derived stem cells of Example 1 according to an embodiment of the present invention. The stem cells were capable of 25 cycles of subculture and underwent 60 cycles of cell division over 20 cycles of subculture;

FIG. 2 shows that the umbilical cord-derived stem cells of Example 2 express embryonic stem cell markers at an RNA level;

FIGS. 3 and 4 are analysis results of mesenchymal stem cell markers according to subculturing of the umbilical cord-derived stem cells of Example 3;

In this regard, the x-axis indicates intensity and the y-axis indicates the number of cells (count). CD markers written on the graphs may be distinguished based on changes in the x-axis and the y-axis;

As shown in FIG. 4, it may be concluded that CD29, CD73, CD105, and CD166, which are properties of mesenchymal stem cells are maintained up to 20 cycles of subculturing but are lost from 25 cycles or more of subculturing;

FIG. 5 is an image showing differentiation potency (differentiation into chondrocytes, osteocytes, and adipocytes) of the umbilical cord-derived stem cell of Example 4;

FIGS. 6 and 7 show analysis results of the maintenance of morphology according to a ratio of collagen to hyaluronic acid derivative in the matrices manufactured in Examples 5 and 7;

FIGS. 8 and 9 show proliferation and survival rates of cells in the matrix manufactured in Example 7 when umbilical cord-derived stem cells are mixed with the matrix;

It may be concluded that an experiment group in which a hyaluronic acid derivative and umbilical cord-derived collagen are mixed shows higher proliferation potency and survival rate than a control group only formed of collagen;

FIG. 10 shows in vivo differentiation (mouse subcutaneous) of the umbilical cord-derived stem cells according to Example 8;

FIG. 11 shows an in vivo differentiation (mouse subcutaneous) of the umbilical cord-derived stem cells according to Example 8 through various staining methods;

FIGS. 12 and 13 show results from damaging an articular cartilage of a knee of a rabbit, up to a subchondral bone, and then preparing a non-treatment group, a group only including a support (a control group), and a group in which umbilical cord-derived stem cells and the support are mixed (an experimental group) and then transplanted, wherein the experimental group showed complete regeneration of cartilage after 8 weeks and 16 weeks of the transplantation;

FIG. 14 shows cartilage (a rabbit joint) regeneration effects of a mixture of the umbilical cord-derived stem cells according to Example 9 and a support through H&E staining; and

FIG. 15 shows cartilage (a rabbit joint) regeneration effects of a mixture of the umbilical cord-derived stem cells according to Example 9 and a support through type II collagen staining.

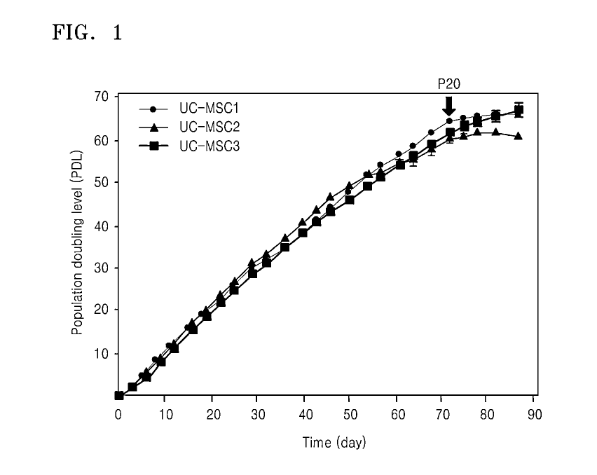

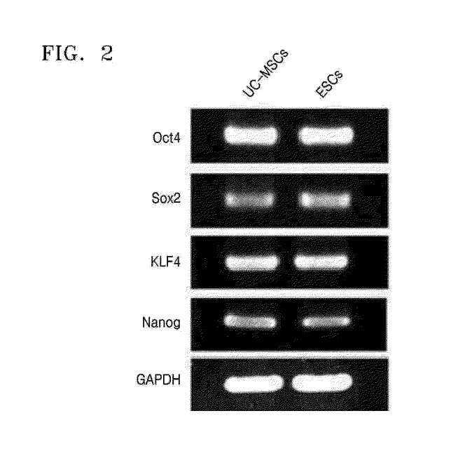

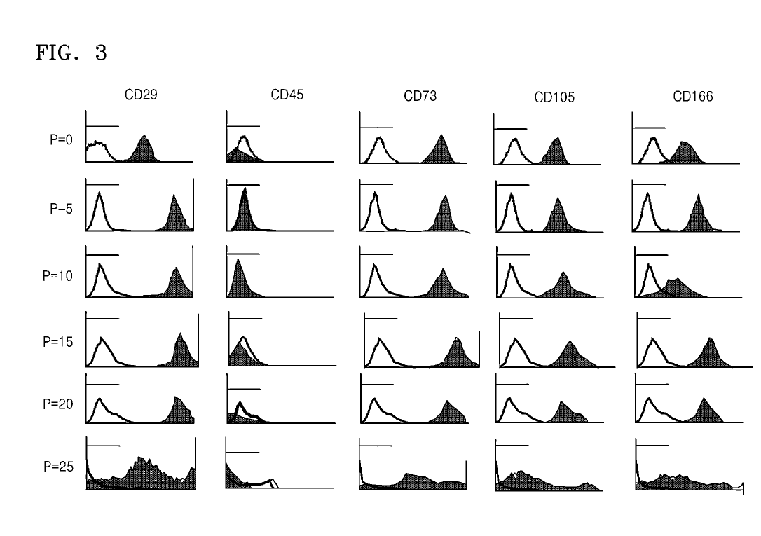

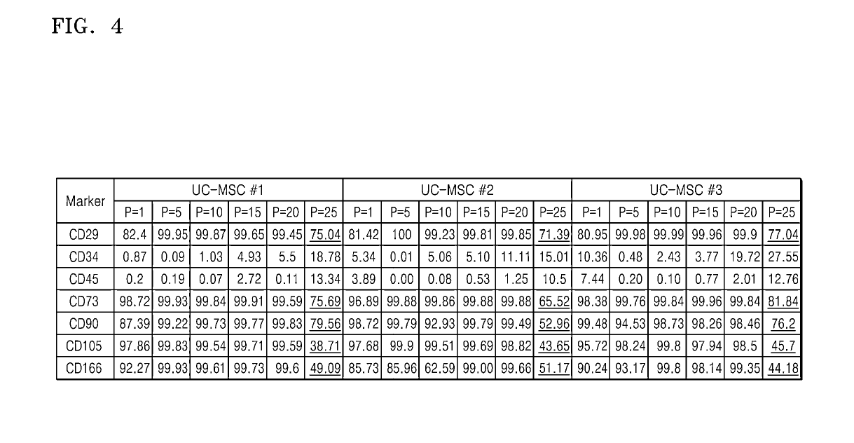

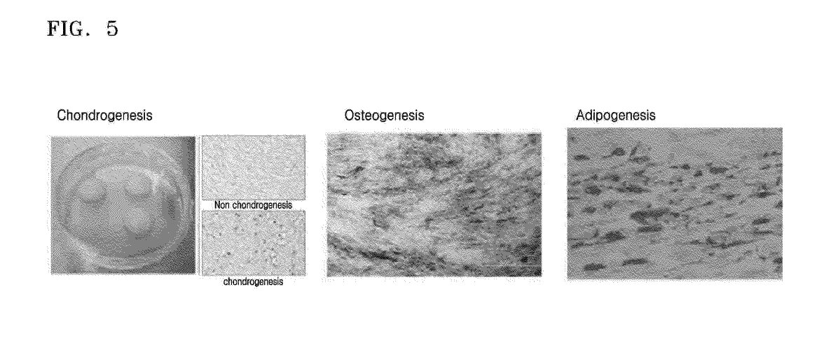

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Provided is a biomaterial composition.

Provided is a composition including collagen and hyaluronic acid or a derivative thereof according to an embodiment.

The collagen may be a mammalian collagen and preferably, a human umbilical cord-derived collagen. Moreover, the human umbilical cord-derived collagen may be a type I collagen. The mammalian collagen may be obtained from various tissues of mammals by using a conventional technology.

More particularly, the human umbilical cord-derived collagen may be prepared by a method including pulverizing human umbilical cord tissue treated with hydrogen peroxide; treating the pulverized human umbilical cord tissue with acetic acid and pepsin and then centrifuging the same; setting a pH of a supernatant obtained from the centrifugation at 7 and adding NaCl thereto to immerse collagen; and separating the immersed collagen.

The hyaluronic acid derivative may be prepared by a method of preparing hyaluronic acid or a salt derivative thereof having excellent biocompatibility and biodegradability, which may be used as a cell carrier of a cell therapy product including stem cells. In this case, the hyaluronic acid derivative may be microparticles.

Furthermore, the hyaluronic acid derivative may be prepared by cross-linking hyaluronic acid using 1,4-butandiol diglycidyl ether (BDDE). Also, the hyaluronic acid derivative prepared for a medical purpose may be pulverized into a micrometer size.

Hyaluronic acid has been known since long ago and is a biocompatible material widely available in nature. Hyaluronic acid is a glycosaminoglycan, which is a necessary component of an extracellular matrix (ECM) and is a linear polysaccharide in which an N-acetylglucosamine monomer and D-glucuronic acid are continuously linked. Hyaluronic acid is a backbone material of biological tissue, which is necessary for morphogenesis, differentiation, and division of cells, and facilitates the recovery of wounds. Hyaluronic acid is a water-insoluble gel in an aqueous solution due to an ether bond but shows excellent elasticity and high moisture absorptive capacity and thus, the hyaluronic acid maintains its shape for a certain period of time in vivo and then decomposes to be absorbed into the body.

Natural hyaluronic acid is rapidly decomposed by a hyaluronidase when the natural hyaluronic acid is injected into the body and thus, the natural hyaluronic acid needs to be cross-linked by various methods or structurally deformed by a chemical material such as benzyl alcohol to control the decomposition speed.

In a medical biomaterial composition according to an embodiment of the present invention, the hyaluronic acid or a salt thereof is not particular limited. The hyaluronic acid or a salt thereof may be added to a basic aqueous solution of about 0.1 N to about 10 N at a concentration of about 1% to about 50%, and a cross-linking agent is added thereto in an amount corresponding to an equivalence ratio of about 0.01% to about 200% based on the amount of the repeating units of the hyaluronic acid or the salt thereof. Preferably, the cross-linking agent is added in an amount corresponding to an equivalence ratio of about 0.1% to about 50% to mix the hyaluronic acid or the salt thereof uniformly to prepare a mixture solution. An amount of time for preparing the mixture solution is not particularly limited, and may preferably be about 1 hour to about 48 hours.

The cross-linking agent including two or more epoxy functional groups is not particularly limited, but preferable examples may include compounds such as 1BDDE, ethylene glycol diglycidyl ether (EGDE), 1,6-hexanediol diglycidyl ether, propylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, trimethylpropane polyglycidyl ether, 1,2-(bis(2,3-epoxypropoxy)ethylene, pentaerythritol polyglycidyl ether, and sorbitol polyglycidyl ether.

The mixture solution is reacted and then washed with a saline solution to remove unreacted products. The product obtained therefrom is pulverized into a micro size by using a pulverizer and then washed with a saline solution. The washed product obtained therefrom is pulverized to adjust the particle size thereof, and the concentration thereof is adjusted to preferably about 0.5% to about 10%, and more preferably about 1% to about 3%. Thereafter, the pulverized product obtained therefrom is autoclaved at a temperature of about 100° C. or greater and preferably about 121° C. or greater to prepare hyaluronic acid derivative that may finally be applied to a human body as a medical biomaterial composition.

The cross-linked hyaluronic acid derivative is a hydrogel-type material that has a net structure and thus, when the cross-linked hyaluronic acid derivative contacts surrounding water molecules, the cross-linked hyaluronic acid derivative swells and the volume thereof increases. However, a gel-type collagen shrinks under the same condition unlike the hyaluronic acid and thus, the volume thereof decreases. However, according to the method developed in the present invention, when the hyaluronic acid or the hyaluronic acid derivative is mixed at a specific ratio with collagen to prepare a composition, the composition and stem cells may be mixed such that swelling or shrinking may not occur when the stem cells are cultured in vitro.

A mixture ratio of the hyaluronic acid derivative and a mammalian umbilical cord-derived collagen may be about 1:10 to about 10:1, about 1:5 to about 5:1, and may preferably be about 1:1 to about 1:3. Also, the mammalian umbilical cord-derived stem cells may be mixed with the hyaluronic acid derivative and the mammalian umbilical cord-derived collagen gel at a concentration of about 1.0×10⁴ to about 1.0×10¹¹ cells/ml, about 1.0×10⁵ to about 1.0×10⁹ cells/ml, and preferably at about 1.0×10⁶ to about 1×10⁷ cells/ml.

When the medical composite biomaterial of the present invention is implanted into the body, surrounding human tissue cells move into the medical composite biomaterial. The human tissue cells secrete extracellular material and thus, even when the medical composite biomaterial degrades, the extracellular material maintains desired effects. In this regard, the medical biomaterial composition shows results that may not be anticipated from the conventional art.

According to another aspect of the present invention, provided is a cartilage cell treatment including stem cells, collagen, and hyaluronic acid or a derivative thereof.

In the cartilage cell treatment of the present invention, the stem cells may be mammalian stem cells. The mammalian umbilical cord-derived stem cells include a mammalian umbilical cord extract and a stem cell culture medium composition for separating or culturing the mammalian umbilical cord-derived stem cells, which may be prepared by a continuous subculturing in a container coated with cell adhesion proteins. In this regard, the stem cell culture medium may not include blood serum.

Also, the mammalian umbilical cord-derived stem cells may be prepared by a method of separating stem cells from a mammalian umbilical cord, the method including (i) adding a cell culture medium composition for separating or culturing mammalian umbilical cord-derived stem cells including a mammalian umbilical cord extract and no serum to remove blood from a container coated with cell adhesion proteins and adding morcellated mammalian umbilical cord tissues thereto to culture the same; and (ii) treating the cultured product obtained therefrom with a medium containing a stem cell separation enzyme to separate the mammalian umbilical cord-derived stem cells. Also, the mammal may be a human, pig, horse, cow, mouse, rat, hamster, rabbit, goat, or sheep.

Also, the mammalian umbilical cord extract may be prepared by a method of preparing a mammalian umbilical cord extract, the method including (i) putting a morcellated umbilical cord into a buffer to stir the same to obtain a solution and (ii) recovering a supernatant of the solution obtained from process (i).

In the method of separating stem cells from the mammalian umbilical cord, wherein the stem cell separation enzyme in process (ii) may be a collagenase, preferably, a type I collagenase and (ii) an amount of the type I collagenase may be about 180 U/ml to about 220 U/ml, which may be treated for about 2 hours to about 6 hours. Also, process (ii) may be performed about 1 day to about 10 days after starting process (i).

Also, the cell adhesion protein may be, but is not limited to mammalian umbilical cord-derived collagen, gelatin, fibronectin, laminin, or poly-D-lysin. Furthermore, the mammalian umbilical cord-derived stem cells may be human umbilical cord-derived stem cells.

In the cartilage cell treatment according to an embodiment of the present invention, the collagen may be obtained from a mammalian umbilical cord. The collagen may be obtained by various methods. However, the mammalian umbilical cord-derived collagen may preferably be prepared by a method including pulverizing mammalian umbilical cord tissues treated with hydrogen peroxide; treating the pulverized mammalian umbilical cord tissues with acetic acid and pepsin and centrifuging the same; setting a pH of a supernatant obtained from the centrifugation at 7 and adding NaCl thereto to immerse collagen; and separating the immersed collagen.

In the cartilage cell treatment according to an embodiment of the present invention, the hyaluronic acid derivative may be prepared by a method of preparing hyaluronic acid or a salt derivative thereof having excellent biocompatibility and biodegradability, which may be used as a cell carrier of cell therapy product including stem cells. In this case, the hyaluronic acid derivative may be microparticles.

Furthermore, the hyaluronic acid derivative may be obtained by cross-linking hyaluronic acid derivative using BDDE. Also, the hyaluronic acid derivative prepared for a medical purpose may be pulverized into a micrometer size.

In the cartilage cell treatment according to an embodiment of the present invention, the hyaluronic acid or a salt thereof is not particular limited. The hyaluronic acid or a salt thereof may be added to a basic aqueous solution of about 0.1 N to about 10 N at a concentration of about 1% to about 50%, and a cross-linking agent is added thereto in an amount corresponding to an equivalence ratio of about 0.01% to about 200% based on the amount of the repeating units of the hyaluronic acid or the salt thereof. Preferably, the cross-linking agent is added in an amount corresponding to an equivalence ratio of about 0.1% to about 50% to uniformly mix the hyaluronic acid or the salt thereof to prepare a mixture solution. An amount of time for preparing the mixture solution is not particularly limited, and may preferably be about 1 hour to about 48 hours.

After reacting the mixture solution, the mixture solution is washed with a saline solution. The washed product obtained therefrom is pulverized into a micro size to adjust the particle size thereof, and the concentration thereof is adjusted to about 1% to about 3%. Thereafter, the pulverized product obtained therefrom is autoclaved at a temperature of about 100° C. or greater and preferably about 121° C. or greater to prepare a hyaluronic acid derivative that may finally be applied to a human body, which may be used for the cartilage cell treatment according to an embodiment of the present invention.

In the cartilage cell treatment according to an embodiment of the present invention, a mixture ratio of the hyaluronic acid derivative and the mammalian umbilical cord-derived collagen may be about 1:10 to about 10:1, about 1:5 to about 5:1, and may preferably be about 1:1 to about 1:3. Also, the mammalian umbilical cord-derived stem cells may be mixed with the hyaluronic acid derivative and the mammalian umbilical cord-derived collagen gel at a concentration of about 1.0×10⁴ to about 1.0×10″ cells/ml, about 1.0×10⁵ to about 1.0×10⁹ cells/ml, and preferably at about 1.0×10⁶ to about 1×10⁸ cells/ml.

The cartilage cell treatment according to an embodiment of the present invention may preferably have a hydrogel form. As a result, the cartilage cell treatment may be easily injected into a damaged cartilage area. Also, the cartilage cell treatment may be used as a composition for a cartilage cell treatment injection including the cartilage cell treatment.

The wording “composition for injection or cell therapy for injection” refers to a pharmaceutical composition that may be injected into a defective area or an adjacent area thereof through a parenteral injection such as a needle injection including stem cells to correct defects of a tissue.

Depending on the form thereof, a suspending agent, a solubilizer, a stabilizer, an isotonic agent, a preservative, an adsorption preventing agent, a surface active agent, a diluent, an excipient, a pH adjuster, a soothing agent, a buffering agent, a sulfur-containing reducing agent, an antioxidant, or the like may be suitably added thereto, as needed.

Examples of the suspending agent include methyl cellulose, polysorbate 80, hydroxyethyl cellulose, gum Arabic, tragacanth gum, sodium carboxymethyl cellulose, and polyoxyethylene sorbitan monolaurate.

Examples of the solubilizer include polyoxyethylene hardened castor oil, polysorbate 80, nicotinamide, polyoxyethylene sorbitan monolaurate, macro bone, castor oil fatty acid ethyl ester, and the like.

Examples of the stabilizer include dextran 40, methylcellulose, gelatin, sodium sulfite, and sodium meta sulfate.

Examples of the isotonic agent include D-mannitol and sorbitol.

Examples of the preservative include p-methyl benzoate, ethyl p-benzoate, sorbic acid, phenol, cresol, and chloro-cresol.

Examples of the adsorption preventing agent include human serum albumin, lecithin, dextran, an ethylene oxide propylene oxide copolymer, hydroxypropyl cellulose, methylcellulose, polyoxyethylene hydrogenated castor oil, and polyethylene glycol.

Examples of the sulfur-containing reducing agent include N-acetyl cysteine, N-acetyl homocysteine, thioctic acid, thiodiglycol, triethanolamine, thio glycerol, thio sorbitol, thioglycolic acid and a salt thereof, sodium thiosulfate, glutathione, and a compound including a sulfhydryl group such as C1-C7 thio alkane acid.

Examples of the antioxidant include chelating agents such as erythorbic acid, dibutylhydroxytoluene, butylhydroxyanisole, α-tocopherol, tocopherol acetate, L-ascorbic acid and a salt thereof, L-ascorbic acid palmitate, L-ascorbic acid stearate, sodium hydrogen sulfite, sodium sulfite, garlic acid triamyl, garlic acid propyl or ethylenediaminetetraacetic acid sodium (EDTA), sodium pyrophosphate, and sodium metaphosphate.

Injection products according to embodiments of the present invention may be prepared as injections filled with amounts conventionally known in the art according to the conditions of and the type of defect of a patient.

The injection products according to embodiments of the present invention may be injected into defective areas or areas adjacent thereto.

According to another aspect of the present invention, provided is a method of treating damaged cartilage including injecting the cartilage cell treatment injection to a patient.

The composition for injection may be directly injected to a damaged cartilage of a patient or an area adjacent to the damaged cartilage. More particularly, the damaged cartilage may be treated without surgery, by using arthroscopy.

### Example 1

### Analysis of Separation and Proliferation Potency of Umbilical Cord Stem Cells

The umbilical cords used in the present research were umbilical cords discarded after delivery by normal mothers upon their agreement. The umbilical cords were used within 24 hours after collecting the umbilical cords.

Tissues removed of blood external to the umbilical cord by using DPBS without Ca²⁺ and Mg²⁺ were removed of an external amnion and two arteries, cut into a size of 1 mm³, and then put into α-minimum essential medium (α-MEM) including 100 U/mL of penicillin, 0.1 μg/mL of streptomycin, and 0.2 μg/mL of an umbilical cord extract. After culturing the same for 7 days, when cells appeared to adhere to the bottom, the cells were treated with 500 U/ml of α-MEM including type I collagenase for four hours to separate the cells. Thereafter, 2×10³ of the cells were inoculated per 1 cm² of a culture dish including α-MEM in which 100 U/ml of penicillin, 0.1 μg/ml of streptomycin, and 0.2 mg/ml of the umbilical cord extract were included, to culture the cells in an incubator in which 5% of CO₂ was supplied at a temperature 37° C. The cell culture medium was replaced three times per week and when the cells grew up to about 70% to about 80% in a cell culturing container, the cells were treated with 0.125% of trypsin and 1 mM of EDTA for three minutes to detach the cells, and 2×10³ of the cells were disposed per 1 cm² of the cell culture dish and then cultured in an incubator in which 5% of CO₂ was supplied.

### Example 2

### Analysis of Embryonic Stem Cell Markers Through RT-PCR

Cell pellets were washed with DPBS without Ca²⁺ and Mg²⁺, 1 ml of lysis buffer (a product of iNtRON Biotechnology) was added thereto, and then a total RNA was separated therefrom according to the method described in the manual available from iNtRON Biotechnology. 1 μg of RNA was reverse transcribed by using a cDNA synthesis kit (a product of iNtRON Biotechnology) in a 20 μL of a reaction solution including a reaction buffer, 1 mM of dNTP mixture, 0.5 μg/μL of oligo(dT)15, 20 U of RNase inhibitor, and 20 U of AMV reverse transcriptase. The reaction was performed at a temperature of 42° C. for 60 minutes. The RT products (cDNAs) obtained therefrom were subjected to PCR by using a 2×PCR Master mix solution kit (a product of iNtRON Biotechnology) including 10 μL of a reaction solution including 1× Taq buffer, 0.25 U of Taq polymerase, and 10 pM of sense and antisense gene-specific primers. The amplification was performed for a total of 32 cycles and each cycle included 30 seconds of denaturation at a temperature of 94° C., 30 seconds of annealing, and 30 seconds of extension at a temperature of 72° C. After completing the reaction, the PCR products obtained therefrom were loaded in a 2% agarose gel for electrophoresis. After the electrophoresis, the gel was stained with ethidium bromide and a DNA image was obtained by using ultraviolet rays.

TABLE 1
DNA sequence information of primers

| | | | | Temperature |
|---|---|---|---|---|
| Genes | | Primer sequences (5′-3′) | | (° C.) |
| OCT 4 | Sense | agaaggagtggtccgagtg | SEQ ID NO: 1 | 60 |
| | Antisense | agagtggtgacggagacagg | SEQ ID NO: 2 | |
| Nanog | Sense | atacctcagcctccagcaga | SEQ ID NO: 3 | 59 |
| | Antisense | cctgattgrrccaggattgg | SEQ ID NO: 4 | |
| KLF4 | Sense | accctgggtcttgaggaagt | SEQ ID NO: 5 | 59 |
| | Antisense | tgccttgagatgggaactct | SEQ ID NO: 6 | |
| Sox2 | Sense | gatgcacaactcggagatcag | SEQ ID NO: 7 | 60 |
| | Antisense | gccgttcatgtaggtctgcga | SEQ ID NO: 8 | |
| GAPDH | Sense | gaaggtgaaggtcggagtca | SEQ ID NO: 9 | 60 |
| | Antisense | ggaggcattgctgatgatct | SEQ ID NO: 10 | 60 |

### Example 3

### Expression Analysis of Mesenchymal Stem Cell Markers Through FACS Analysis

A flow cytometry was used to analyze properties of separated cells. The separated cells were washed by using PBS, treated with trypsin-EDTA to make a monoclonal cell group, and then washed with PBS including 2% FBS and 1 mM EDTA. Thereafter, stem cells markers bound to fluorescein isothiocyanate (FITC) or phycoerythrin (PE) were treated, left to react for 20 minutes and then analyzed by using FACSCalibur (a product of Becton-Dickinson).

### Example 4

### Analysis of Differentiation Potency of Umbilical Cord Stem Cells

<4-1> Induction of Differentiation into Adipocytes

2×10³ of umbilical cord-derived stem cells per 1 cm² were put into of 24-well plate, cultured for 3 days, and then a cell culture medium was replaced with a cell differentiation medium in which 10% of FBS, 1 μM of dexamethasone, 0.5 μM of 3-3-isobutyl-1-methylxanthine, 0.05 mg/L of human insulin, and 200 μM of indomethacin were included in DMEM. Then, the cell culture medium was replaced 3 times per week. After culturing for 3 weeks, Oil Red 0 staining was performed to analyze the presence of adipocytes with lipids accumulated therein.

<4-2> Induction of Differentiation into Osteocytes

2×10³ of umbilical cord-derived stem cells per 1 cm² were put into a 24-well plate, cultured for 3 days, and then a cell culture medium was replaced with a cell differentiation medium including 10% of FBS, 0.1 μM of dexamethasone, 100 mM of 3-glycerol phosphate, and 50 μM of ascorbic acid-2-phosphate. Then, the cell culture medium was replaced 3 times per week. After culturing for 3 weeks, ALP staining was performed to analyze differentiation.

<4-3> Induction of Differentiation into Cartilage Cells

2×10³ of umbilical cord-derived stem cells per 1 cm² were mixed with a collagen gel to inoculate 100 μl of the same to a culturing dish, maintained for 10 minutes in an incubator at a temperature of 37° C. to gelate collagen, and a cell differentiation medium including 0.1 μM of dexamethasone, 50 μg/mL of ascorbic acid-2-phosphate, 100 μg/mL of sodium pyruvate, 10 ng/mL of transforming growth factor-β3, and 50 mg/mL of ITS plus premix (including 6.25 μg/mL of insulin, transferrin, and selenious acid) in DMEM were added thereto to culture the umbilical cord-derived stem cells. Then, the cell culture medium was replaced three times per week while only a half of the total volume was replaced with a new medium. After culturing for three weeks, type II collagen staining was performed to analyze differentiation.

### Example 5

### Preparation of Hyaluronic Acid Derivative

Hyaluronic acid sodium was dissolved at a concentration of 100 mg/ml in a 0.25 N NaOH solution to obtain a solution. BDDE was added to the solution. After reacting at a temperature of 30° C. for 36 hours, a product obtained therefrom was washed with a saline solution to remove unreacted products. The washed products obtained therefrom were pulverized to adjust the size thereof and the concentration thereof was adjusted to 20 mg/ml to prepare a hyaluronic acid derivative.

### Example 6

### Preparation of Human Umbilical Cord-Derived Collagen

A frozen umbilical cord was thawed at room temperature. The umbilical cord was cut into a length of about 1 cm to about 2 cm and then washed with distilled water. The umbilical cord was washed with a 70% ethanol solution and then reacted at 4° C. for 24 hours. Then, the umbilical cord was washed with distilled water, treated with a 3% H₂O₂ solution and then stirred at 4° C. for about 12 hours to about 24 hours by using a magnetic bar. Then, the product obtained therefrom was washed twice or more with distilled water and tissues were pulverized after adding a 0.5 M acetic acid solution and by using a blender and a homogenizer. Then, the pulverized product obtained therefrom was treated with pepsin and reacted at a temperature of 4° C. for 24 hours and then centrifuged at 10,000 rpm at 4° C. for 30 minutes.

After the centrifugation, a pH of the supernatant obtained therefrom was set at 7 by using NaOH to remove activities of pepsin. The pH-adjusted solution obtained therefrom was treated with NaCl, stirred until NaCl was completely dissolved, and then maintained at 4° C. for about 12 hours to about 24 hours until collagen salted out and immersed. The immersed product obtained therefrom was centrifuged at 10,000 rpm, at 4° C. for 30 minutes, the salted out collagen pellets obtained therefrom were separated to be desalted and concentrated by using an ultrafiltration system. Finally, the desalted and concentrated product obtained therefrom was removed of microorganisms, freeze-dried, and then stored to prepare a collagen solution. The collagen solution was quantified through hydroxyprolin analysis and the purity thereof was analyzed through SDS-PAGE.

### Example 7

### Preparation of a Composite Hydrogel and 3-Dimensional in Vitro Culturing

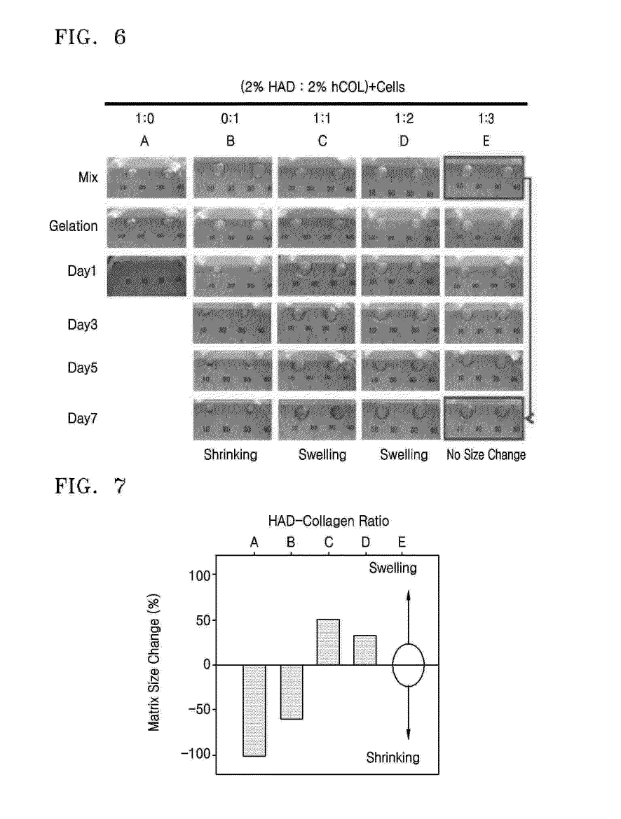

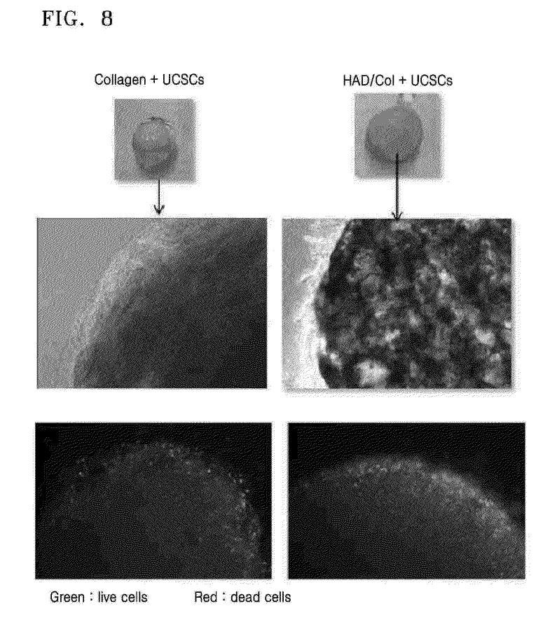

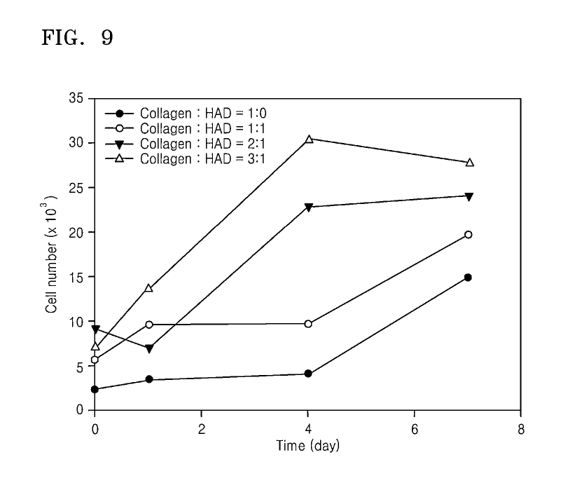

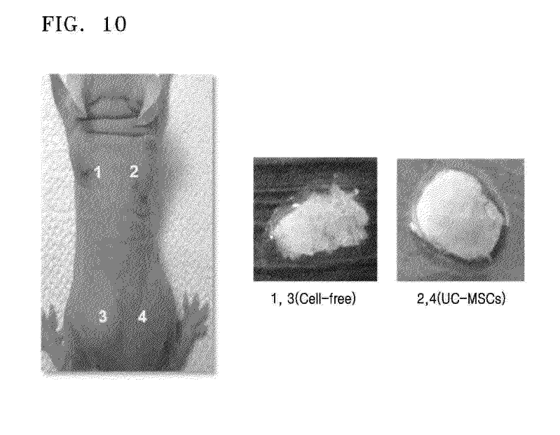

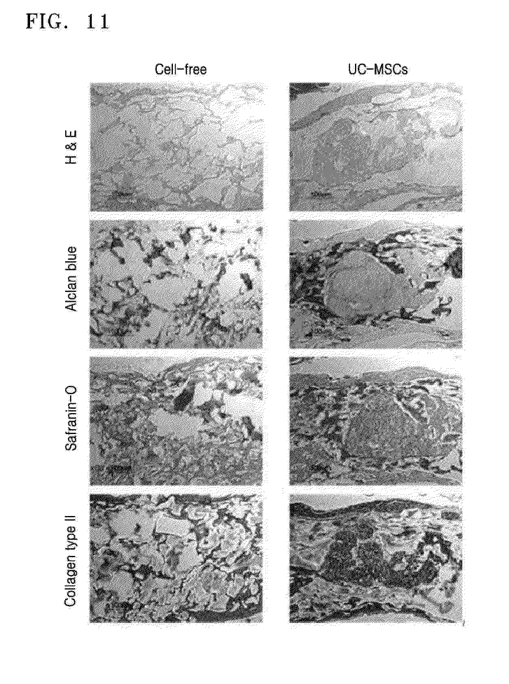

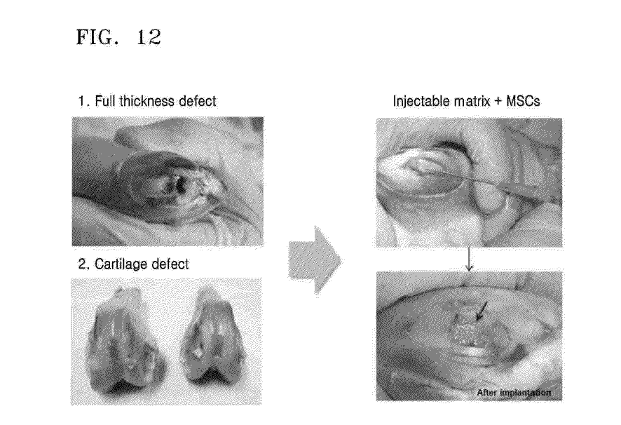

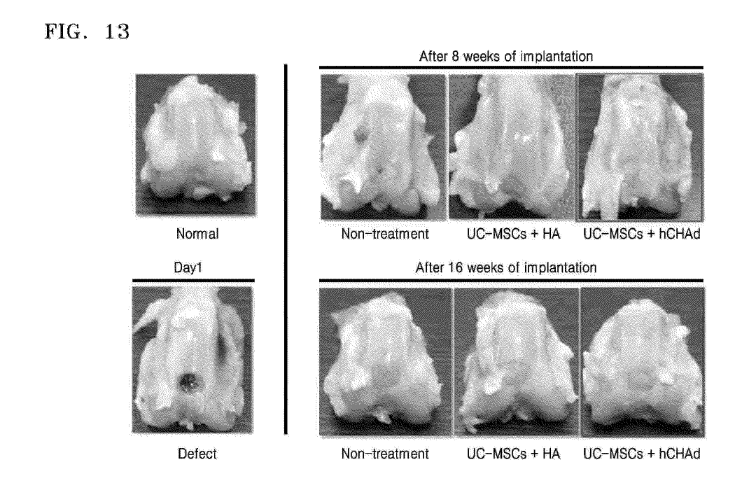

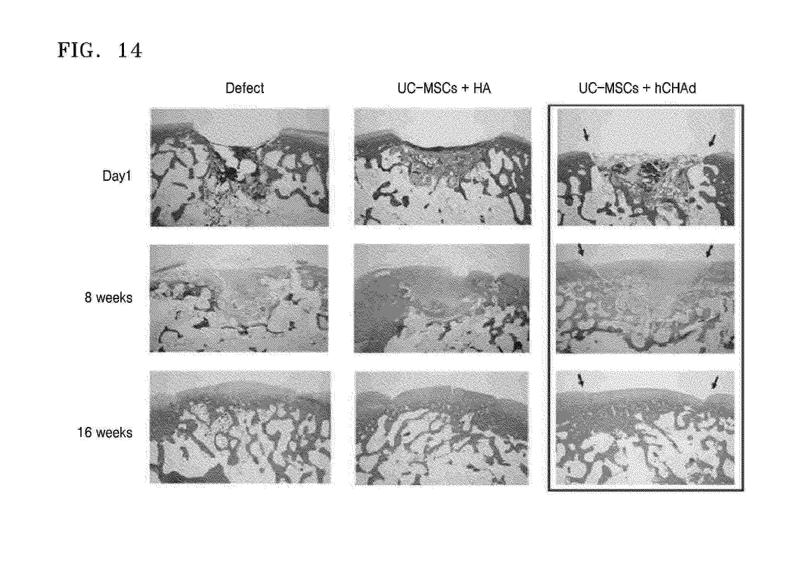

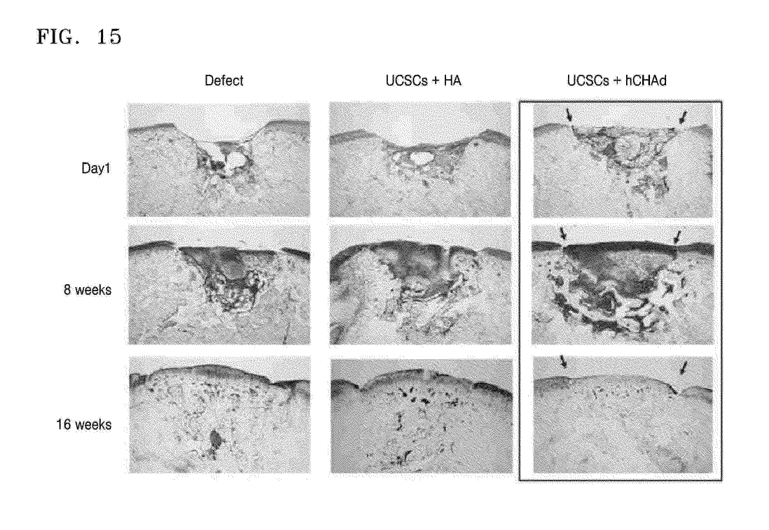

To determine an optimal mixture ratio of a 2% (w/v) hyaluronic acid derivative and the 3% (w/v) collagen, the two materials and stem cells were mixed at volume ratios under various conditions and then changes in volume were analyzed in a cell culture medium (FIGS. 6 and 7). A ratio at which a volume of a composite hydrogel does not change with culturing time was selected to prepare the final composite hydrogel.

A hydrogel including the two materials was prepared at an optimal ratio, then a buffer solution including NaHCO₃ and HEPES dissolved in 0.05 N NaOH solution and cells were mixed, and then the cells were divided into an amount of about 20 μl to about 40 μl in culturing containers. The cells were put into an incubator at a temperature of 37° C. in which 5% of CO₂ was supplied for 20 minutes. Then, when the matrix including the cells became opaque, a cell culture medium was added thereto to culture the cells and the cell culture medium was replaced every three days.

### Example 8

### Differentiation Potency In Vivo (by Using a Mouse Model)

A rat (BALB/c-nu Slc) used for the present experiment was a female about 5 weeks old. For each experimental group, 100 μl of an injection including 10 ng of TGF-β3 was subcutaneously injected to the rat and a sample was obtained from the rat after 4 weeks. The sample was fixed with 4% neutral buffered formalin and the extent of cartilaginification was analyzed through Hematoxylin & Eosin (H&E), Alcian blue, Safranin-O staining and type II collagen immune staining.

### Example 9

### Analysis of Effects In Vivo (a Rabbit Joint Model)

A female New Zealand white rabbit that weighed about 3 kg to about 3.5 kg was used for the present experiment was. The rabbit was anesthetized, a knee of the rabbit was cut to damage a knee joint area, the area having a radius of 2.5 mm up to a subchondral bone. As control groups, a group that was not treated at all and a group only treated with a support were used. As an experimental group, umbilical cord stem cells and the support were transplanted together to perform the experiment. Samples were extracted after 8 weeks and 16 weeks, the samples were fixed by using 4% neutral buffer formalin and then subjected to H&E staining and type II collagen immune staining to analyze the effects of cartilage regeneration.

The composition including collagen and hyaluronic acid derivative according to an embodiment of the present invention may be used as a medical composite biomaterial for various purposes. Also, when stem cells are added thereto, the composition may be used as an effective cartilage treatment. A conventional cartilage treatment is mostly a surgical treatment; however, when the composition according to an embodiment of the present invention is used, the cartilage treatment is possible without surgery. Thus, the composition has high industrial applicability.

As described above, according to the one or more of the above embodiments of the present invention, the medical composite biomaterial includes a composition similar to human skin tissues, in other words, includes human collagen and hyaluronic acid derivative. Thus, the medical composite biomaterial has excellent affinity to human cells. Also, instead of surgery, an anthroscope may be used for a simple treatment and the composition may be easily transplanted by injection to damaged cartilage tissues, wherein the composition becomes fixed after gelation.

The cartilage cell treatment according to an embodiment of the present invention has a hydrogel form, which has a slower rate of decomposition than a conventional support only using hyaluronic acid. Thus, the cartilage cell treatment may maintain its shape despite external physical and mechanical effects to maintain excellent treatment effects for a long time, and may provide an excellent cartilage cell treatment.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

What is claimed is:

1. A composition comprising collagen and hyaluronic acid or a hyaluronic acid derivative.

2. The composition of claim 1, wherein the collagen is mammalian collagen.

3. The composition of claim 2, wherein the mammalian collagen is human umbilical cord-derived collagen.

4. The composition of claim 3, wherein the human umbilical cord-derived collagen is type I collagen.

5. The composition of claim 1, wherein the hyaluronic acid derivative is prepared by cross-linking hyaluronic acid using 1,4-butandiol diglycidyl ether (BDDE).

6. A medical composite biomaterial comprising the composition of claim 1.

7. A cartilage cell treatment comprising stem cells, collagen, and hyaluronic acid or a hyaluronic acid derivative.

8. The cartilage cell treatment of claim 7, wherein the stem cells are mammalian stem cells.

9. The cartilage cell treatment of claim 8, wherein the mammalian stem cells are umbilical cord-derived stem cells.

10. The cartilage cell treatment of claim 7, wherein the collagen is obtained from a mammalian umbilical cord.

11. The cartilage cell treatment of claim 7, wherein the stem cells are cultured by using a stem cell culture medium comprising a mammalian umbilical cord extract in a container coated with cell adhesion proteins.

12. The cartilage cell treatment of claim 11, wherein the stem cell culture medium does not include serum.

13. The cartilage cell treatment of claim 11, wherein the cell adhesion protein is selected from the group consisting of a mammalian umbilical cord-derived collagen, gelatin, fibronectin, laminin, and poly-D lysin.

14. The cartilage cell treatment of claim 7, wherein the hyaluronic acid derivative is prepared by cross-linking hyaluronic acid by using 1,4-butandiol diglycidyl ether (BDDE).

15. The cartilage cell treatment of claim 7, wherein a mixture ratio of hyaluronic acid or a hyaluronic acid derivative and the collagen is about 3:1 to about 1:3.

16. The cartilage cell treatment of claim 15, wherein the hyaluronic acid or the hyaluronic acid derivative is comprised in an amount of about 1% to about 3%.

17. The cartilage cell treatment of claim 15, wherein the collagen is comprised in an amount of about 1% to about 3%.

18. The cartilage cell treatment of claim 7, wherein the mammalian umbilical cord-derived stem cells are mixed with hyaluronic acid and mammalian umbilical cord-derived collagen at a concentration of about 1.0×10⁶ to about 1×10⁸ cells/ml.

19. The cartilage cell treatment of claim 7, wherein the cartilage cell treatment is hydrogel.

20. A cartilage cell treatment injection comprising the cartilage cell treatment of claim 7.

21. A method of treating damaged cartilage comprising injecting the cartilage cell treatment injection of claim 20 to a patient.
